(19) United States
(12) Patent Application Publication
(10) Pub. No.: US 2021/0065901 A1
(43) Pub. Date: Mar. 4, 2021

(54) SYSTEM AND METHOD OF DETERMINING A CONDITION OF A SUBJECT BASED ON VOLATILE ORGANIC COMPOUNDS

(71) Applicant: NanoScent Ltd., Mishgav (IL)

(72) Inventors: Oren GAVRIELY, Yaad, Misgav (IL); Orna BARASH, Kiryat Tivon (IL); Eran ROM, Yaad.Misgav (IL); Jacob BLANK, Ramat Hasharon (IL); Shir ALONI, Yuvalim, Misgav (IL); Yair PASKA, Kiryat Ata (IL)

(73) Assignee: NanoScent Ltd., Mishgav (IL)

(21) Appl. No.: 16/960,094

(22) PCT Filed: Jan. 3, 2019
(86) PCT No.: PCT/IL2019/050017

§ 371 Date: Jul. 6, 2020

Related U.S. Application Data
(60) Provisional application No. 62/613,426, filed on Jan. 4, 2018.
(60) Provisional application No. 62/613,428, filed on Jan. 4, 2018.
(60) Provisional application No. 62/643,434, filed on Mar. 15, 2018.
(60) Provisional application No. 62/613,435, filed on Jan. 4, 2018.
(60) Provisional application No. 62/651,740, filed on Apr. 3, 2018.
(60) Provisional application No. 62/683,314, filed on Jun. 11, 2018.

(51) Int. Cl. G16H 50/20; A61B 10/00; G16H 50/70; G16H 10/60; G16H 10/40; G16H 10/20; G16H 40/67; G01N 33/497; G01N 33/487

(52) U.S. Cl. CPC G16H 50/20; A61B 10/00; G16H 50/70; G16H 10/60; G16H 10/40; G16H 10/20; G01N 2033/4975; G01N 33/497; G01N 33/487; A61B 2010/0083; A61B 2010/0087; A61B 2560/0431; G16H 40/67

(57) ABSTRACT

Disclosed is a method of determining a condition of a subject based on Volatile Organic Compounds (VOCs) in a gaseous phase, originating from the subject. The method may include: receiving from one or more sensors a set of sensor signals in response to exposing the one or more sensors to a sample of the VOCs; extracting one or more feature values from the set of sensor signals; receiving a classification model, trained to classify samples of VOCs based on the one or more extracted feature values correlated to one or more condition of the subject; associating the one or more extracted features received from the set of sensor signals with one or more classes of the classification model; and determining the condition of the subject based on the association.

### FIELD OF THE INVENTION

The present invention generally relates to determining a condition of a subject based on biometric data. More particularly, the present invention relates to systems and methods of determining a condition of a subject based on biometric data collected from sensors for sensing volatile organic compounds (VOCs) originated from the subject.

### BACKGROUND OF THE INVENTION

Collecting biometric data of subjects includes many widely used methods, such as, image recognition of faces (also kwon as face recognition), finger prints, iris recognition, voice recognition and the like. All the listed methods detect measured parameters and compare these parameters to a measurement of the parameters stored in a database and associated with a single subject. Usually, a single measurement of the subject is enough for collecting all the parameters required for identifying the subject. For example, a single image of the face of the subject will suffice for performing face recognition and a single voice recording will suffice for performing voice recognition.

Using VOCs collected from a subject as a recognition method is much more complicated. Humans' and/or mammals' scent is affected by a plurality of parameters, such as, health, gender, diet, mental condition, general wellbeing (e.g., fitness), environmental condition and the like. One's scent can change during a single day, from morning to evening due to a change in one or more parameters effecting one' s scent, for example, the food eaten. Humans and/or mammals producing large number of various VOCs (e.g., odors) every second. The VOCs can be originated from the skin of the subject, hair of the subject, urine, sweat, saliva, feces and practically any material that originated from the subject's body. A Human can produce as much as couple of thousands different chemical compounds (VOCs) just from the skin.

Human's and/or mammal's nose is one of the most sensitive organs in the body with the ability to distinguish between at least 10,000 different VOCs (e.g., smells). Artificial VOCs sensors are much limited and usually based on conducting elements coated/covered by organic ligands. Each type of organic ligand may be configured to be reacted/connected to different types of VOC or a family of VOCs, however can also to some extant react/connect with other VOCs Accordingly, such sensors may be more sensitive to a single VOC or a specific list of VOCs (e.g., a family of VOCs). A list of known VOCs sensors may include: chemi-resistors, metal oxide sensor (MOS), catalytic near IR sensor, photoionization detectors (PID), UV doas\IR open path sensors, portable gas-choromography mass spectrometer (GS-MS), electro-chemical and the like

However, since the smell/odor of a subject may change dramatically within a single day and defiantly over a longer period of time. Furthermore, there is no standard scale for measuring VOCs (e.g., scent). Accordingly, it is practically impossible to recognize a subject based on a single measurement of VOCs originated from the subject, as can be conducted with other biorecognition methods.

Therefore, it is required to develop a system and method that may use VOCs originating from a subject, to be included in a biorecognition method. Such a biorecognition method can be used not just for identifying an individual but also to identify the condition of that individual, for example, a medical condition, mental condition or even the general wellbeing of the individual.

### SUMMARY OF THE INVENTION

Some aspects of the invention may be related to a method of determining a condition of a subject based on Volatile Organic Compounds (VOCs) in a gaseous phase, originating from the subject. In some embodiments, the method may include: receiving from one or more sensors a set of sensor signals in response to exposing the one or more sensors to a sample of the VOCs; extracting one or more feature values from the set of sensor signals; receiving a classification model, trained to classify samples of VOCs based on the one or more extracted feature values correlated to one or more condition of the subject; associating the one or more extracted features received from the set of sensor signals with one or more classes of the classification model; and determining the condition of the subject based on the association.

In some embodiments, the condition may be at least one of: a medical condition of the subject, a mental condition of the subject, an identity of the subject and a general wellbeing of the subject. In some embodiments, the method may further include receiving additional data; associating the received additional data with one or more classes of the classification model; and determining the condition of the subject also based on the additional data association. In some embodiments, the additional data may include sample related data. In some embodiments, the sample related data may include at least one of: humidity level, temperature, geographic location at which the sample was taken, time and date. In some embodiments, the additional data may include subject data. In some embodiments, the subject related data may include at least one of: gender, age, medical condition, ethnicity, culture, lifestyle and diet. In some embodiments, the subject related data may include data related to a specific sample taken form the subject.

In some embodiments, the VOCs may be collected by at least one of: an absorbing material attached to the at least one subject, an absorbing material attached to a device carried by the at least one subject and a container for collecting VOCs evaporating from the at least one subject. In some embodiments, the at least one subject may be a mammal. In some embodiments, the VOCs may include VOCs included in a at least one of: urine of the subject, sweat of the subject and saliva of the subject.

In some embodiments, the subject may be a human and the method may further include: collecting a VOCs sample when the human uses a toilet; an extracting the VOCs from the sample and exposing the one or more sensors to the extracted VOCs.

In some embodiments, the at least one subject may be a female mammal and the condition is fertility. In some embodiments, the VOCs may include VOCs included in at least one of: urine, sweat and saliva of the female mammal. In some embodiments, the VOCs may include VOCs included in the at least one of: skin and hair of the female mammal. In some embodiments, the at least one subject may include at least two humans and wherein the condition is a chance for a successful matching. In some embodiments, the classification model comprises pairs of one or more feature values received from humans' pairs having at least one indication of having a successful matching. In some embodiments, the at least one indication may include at least one of: a relationship lasting more than a predetermined period, number of children, a reported affection and a reported sexual attraction.

In some embodiments, the at least one subject is a human baby and the condition may be general wellbeing. In some embodiments, the VOCs may include VOCs included in at least one of: urine and feces. In some embodiments, the at least one subject may be a mammal and the condition is an identity of the mammal In some embodiments, the method may further include: receiving one or more initial signals from the one or more sensor; associating the initial signals as surrounding background signals; and filtering background noise from the set of sensor signals using the initial signals

Some aspects of the invention may be related to a system for determining a condition of a subject based on Volatile Organic Compounds (VOCs) in a gaseous phase, originating from the subject, the system may include: one or more VOCs sensors configured to detect VOCs originated from the at least one subject; and a controller configured to: receive from the one or more sensors a set of sensor signals in response to exposing the one or more sensors to a sample of the VOCs; extract one or more feature values from the sensor signals; receive a classification model, trained to classify samples of VOCs based on the one or more extracted feature values correlated to one or more condition of the subject; associate the one or more extracted features received from the set of sensor signals with one or more classes of the classification model; and determine the condition of the subject based on the association.

In some embodiments, the system may further include a chamber for holding the one or more sensors; and a gas circulation system for directing VOCs in a gas phase towards the one or more sensors. In some embodiments, the gas circulation system may include at least one of: a fan, a pump, one or more gas monitoring sensors, and one or more valves. In some embodiments, the system may further include a regeneration device for regenerating the one or more sensors. In some embodiments, the regeneration device may include at least one of: a heating element, a vacuum pump and a stream of gas.

In some embodiments, the system may further include one or more additional sensors for detecting a condition of the at least one subject. In some embodiments, the system may further include a holder for holding an absorbing material carrying the VOCs collected from the at least one subject. In some embodiments, the absorbing material may include an absorbing material configured to absorbed VOCs from the subject. In some embodiments, the one or more sensors may include one or more chemi-resistors comprising metallic nanoparticles coated with organic ligands shell, metal oxide sensor (MOS), catalytic near IR sensor, photoionization detector (PID), IR open path sensor, portable gas-chromatography mass spectrometer (GC-MS), electro-chemical sensor and the like.

In some embodiments, the controller may be configured to carry out any one of the methods herein above.

Some aspects of the invention may be related to a method of training a classification model to determine a condition of a subject, the method may include:
a. receiving from one or more sensors a set of sensor signals in response to exposing the one or more sensors to a sample of VOCs originated from the subject;
b. extracting one or more feature values from the sensor signals;
c. tagging the one or more feature values with a class associated with at least one known condition of the subject; and
d. repeating steps (a) through (c) with a different sample, to train the classification model.

In some embodiments, the condition may be at least one of: a medical condition of the subject, a mental condition of the subject, an identity of the subject and a general wellbeing of the subject. In some embodiments, the method may further include: receiving an additional data; and tagging the additional data with the class associated with the known condition of the subject. In some embodiments, the additional data may include sample related data. In some embodiments, the sample related data may include at least one of: humidity level, temperature, geographic location at which the sample was taken, time and date. In some embodiments, the additional data may include subject data. In some embodiments, the subject related data may include at least one of: gender, age, medical condition, ethnicity, culture, lifestyle and diet. In some embodiments, the subject related data may include data related to a specific sample taken form the subject.

In some embodiments, the VOCs may be collected by at least one of: an absorbing material attached to the at least one subject, an absorbing material attached to a device carried by the at least one subject and a container for collecting VOCs evaporating from the at least one subject. In some embodiments, the method may further include: receiving one or more initial signals from the one or more sensor; associating the initial signals as surrounding background signals; and filtering background noise from the set of sensor signals using the initial signals.

Some aspects of the invention may be related to a system for training a classification model to determine a condition of a subject, the system may include: one or more VOCs sensors configured to detect VOCs originated from the at least one subject; a storage unit; and a controller configured to:
e. receive from one or more sensors a set of sensor signals in response to exposing the one or more sensors to a sample of VOCs originated from the subject;
f. extract one or more feature values from the sensor signals;
g. tag the one or more feature values with a class associated with at least one known condition of the subject;
h. repeat steps (a) through (e) with a different sample, to train the classification model; and
i. store the trained classification model in the storage unit.

In some embodiments, the controller may further be configured to: receive at least one known condition of the subject for each sample. In some embodiments, the system may further include a user interface and the controller may be configured to: receive at least one known condition from the user interface. In some embodiments, the system may further include at least one additional sensor and wherein the controller is configured to: receive at least one known condition from a signal received from the additional sensor. In some embodiments, the additional signal may be indicative of the at least one condition. In some embodiments, the controller may be configured to carry out any one of the methods disclosed herein above.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is an illustration of a system for determining a condition of a subject based on VOCs according to some embodiments of the invention;

FIG. 1B is an illustration of a block diagram of a computing device, according to some embodiments of the invention;

FIG. 1C is an illustration of a flowchart of a method of operating the system of FIG. 1A according to some embodiments of the invention;

FIG. 2A is an illustration of a sensor according to some embodiments of the invention;

FIG. 2B is an illustration of a conductive nanoparticle coated with organic ligands according to some embodiments of the invention;

FIG. 2C is a graph showing a signal received from the sensor of FIG. 2A as a function of time according to some embodiments of the invention;

FIG. 3 is an illustration of a system for determining a condition of a subject based on VOCs according to some embodiments of the invention;

FIG. 4 is an illustration of a system for determining a condition of a subject based on VOCs according to some embodiments of the invention;

FIG. 5 is an illustration of a pad that includes an absorbing material for collecting VOCs originated from a cow according to some embodiments of the invention;

FIG. 6 is 5 is an illustration of a pad that includes an absorbing material for collecting VOCs originated from humans according to some embodiments of the invention;

FIG. 7 is a flowchart of a method of determining a condition of a subject based on VOCs according to some embodiments of the invention;

FIG. 8 is a flowchart of a method of training a classification model to determine a condition of a subject according to some embodiments of the invention; and

FIG. 9 shows 8 graphs representing signals received from sensors according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

A system and a method according to some embodiments of the invention may allow using artificial scenting sensors for detecting changes in conditions of a subject or even the identity of the subject. Such sensors may be configured to detect VOCs in a gas phase. The system may use signals produced by these sensors to determine the condition. Since subject's (e.g., a human or a mammal) odors are very sensitive to changes in the subject's condition, even within a single day, a single measurement taken from the subject cannot be the base for any recognition/diagnostic system and method. Furthermore, there is now standard methods for measuring VOCs and the measurements relay heavily on the type and number of VOCs sensors used. Therefore, a system and a method according to some embodiments, may use artificial intelligence (AI) and machine learning (ML) techniques in order to study and analyze signals received from multiple samples taken from each subject or from a group of subjects thus, to train a classification model to further be used for classify or tag new samples taken form the subject or a different subject. The classification model may be used to determine the condition of the subject.

As used herein the word “subject” may include any living being that produces odors (e.g., VOCs). Subject may include: humans, mammals (e g., livestock) or any other animals.

As used herein the word “condition” may refer to any condition, state or parameter related to a living being and is associable to evaporation of VOCs from the living being, for example, medical condition of the subject, a mental condition of the subject, an identity of the subject, successful matching between two subjects and a general wellbeing of the subject.

As used herein the term “Volatile Organic Compound (VOC)” may include any organic compound that may be evaporated form the subject or originated by the subject. The VOCs may be originated from the skin or hair of the subject (e.g., evaporated by sweat), originated from urine, feces, saliva and the like.

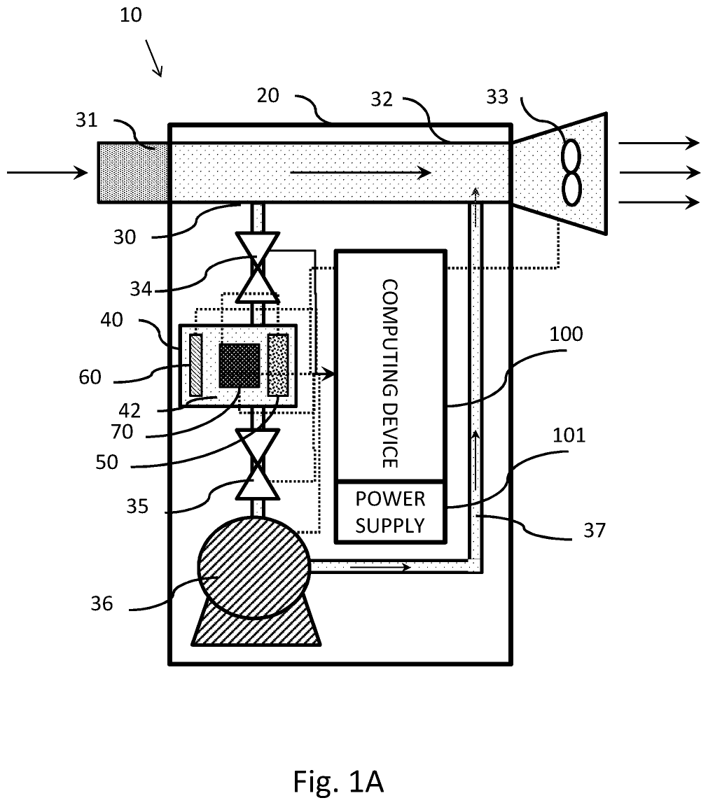

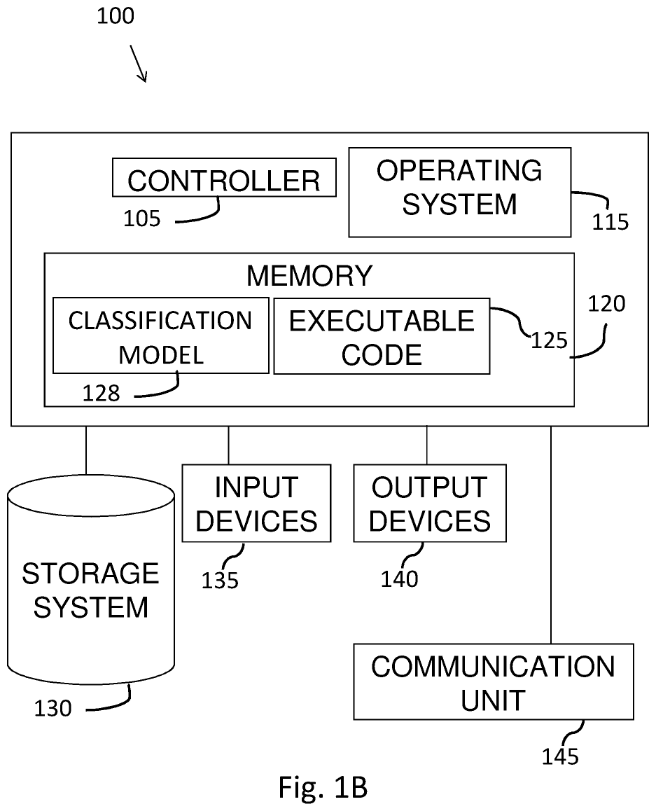

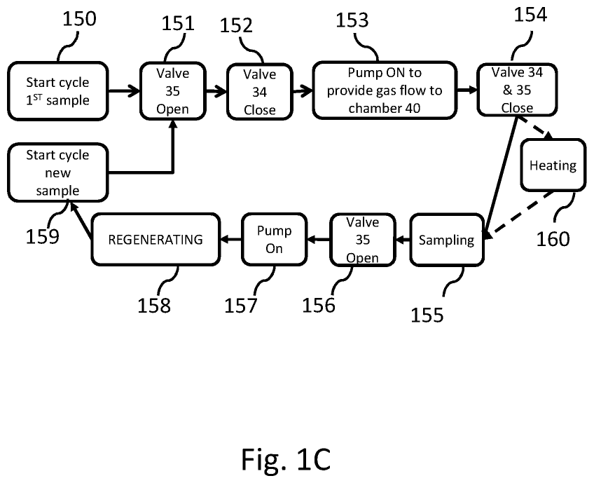

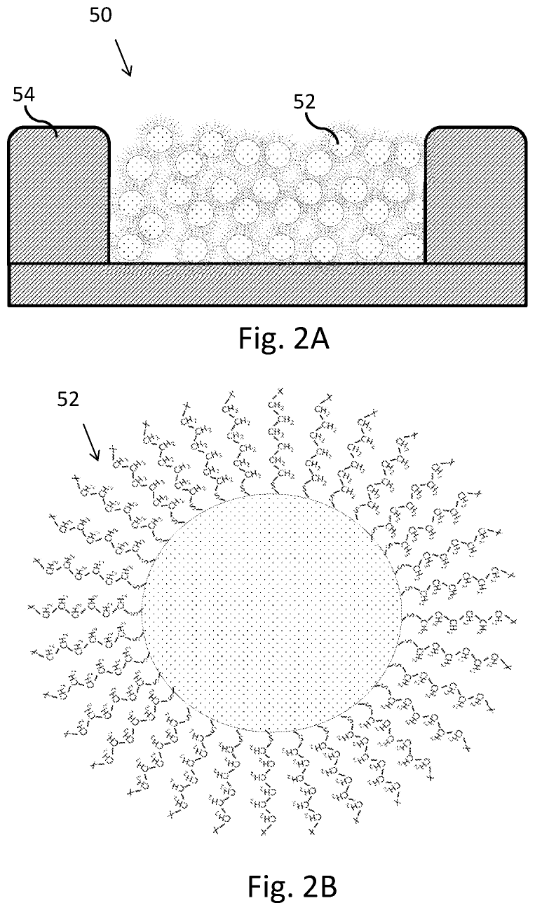

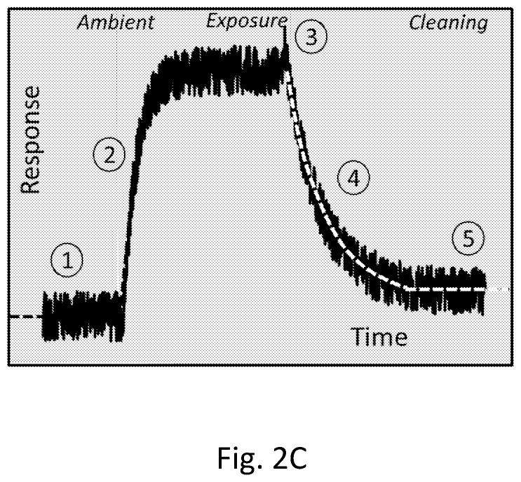

Reference is made to FIG. 1A which is an illustration of a system 10 for determining a condition of a subject based on VOCs in a gaseous phase, originating from the subject according to some embodiments of the invention. . In some embodiments, system 10 may be for training a classification model (e.g., classification model 128 illustrated in FIG. 1B) to determine a condition of a subject. System 10 may include one or more VOCs sensors 50 configured to detect VOCs originated from the at least one subject and a controller 105 of computing device 100 (illustrated and discuused in FIG. 1B). One or more sensors 50 may include any sensor configured to produce a signal (e.g., an electric signal) when exposed to VOCs in the gas phase. For example, one or more sensors 50 may include one or more chemi-resistors comprising metallic nanoparticles coated with organic ligands shell, as discussed in detail with respect to FIGS. 2A-2C. In other examples, one or more VOCs sensors 50 may include at least one of: metal oxide sensor (MOS), catalytic near IR sensor, photoionization detector (PID), IR open path sensor, portable gas-chromatography mass spectrometer (GC-MS), electro-chemical sensor and the like

In some embodiments, system 10 may further include a chamber 40 for holding one or more sensors 50. Chamber 40 may be configured to hold VOCs 42 in the gas phase while exposing one or more sensors 50 of VOCs 42. Chamber 40 may further include one or more additional sensors 60 for sensing an environment and the flow of gases inside chamber 40, for example, a thermometer, a barometer, a humidity sensor, a flowmeter and the like. In some embodiments, chamber 40 may further include a regeneration device 70 for regenerating one or more sensors 50. Regeneration device 70 may include a heating element for heating one or more sensors 50, thus evaporating the VOCs trapped by one or more sensors 50. In some embodiments, regeneration device 70 may include a vacuum pump for causing the evaporating the VOCs due to sub-atmospheric pressure. In some embodiments, regeneration device 70 may include a stream of gas (e.g., clean air or any other gas having a controlled amount of VOCs) to cause flashing of the VOCs from the surface of one or more sensors 50.

In some embodiments, system 10 may further include a gas circulation system 30 for directing VOCs in a gas phase towards the one or more sensors 50 included in chamber 40. Gas circulation system 30 may include: a fan 33, a pump 33, one or more gas monitoring sensors 60, one or more valves 34 and 35, a filter 31, a manifold 32, pipes 37 and the like. In should be understood by one skilled in the art that the component of a gas circulation system 30 illustrated in FIG. 1A are given ad an example only, and a gas circulation system 30 according to embodiments of the invention may include some, all or more of the illustrated components.

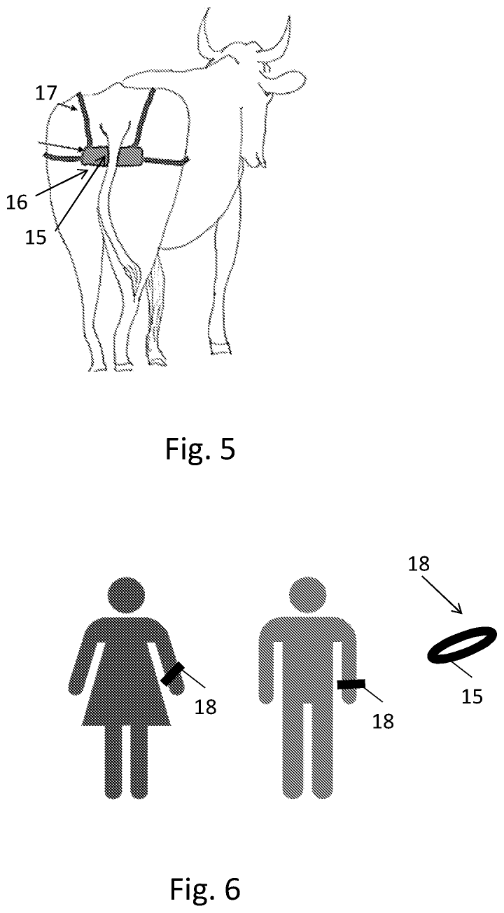

In some embodiments, system 10 may include or may be in fluid connection with at least one holder (not illustrated) for holding an absorbing material carrying the VOCs collected from the at least one subject, as discussed with respect to FIGS. 5 and 6.

Reference is made to FIG. 1B, which is a schematic block diagram of an example of a computing device, according to some embodiments of the invention. Computing device 100 may include a controller or processor 105 (e.g., a central processing unit processor (CPU), a graphics processing unit (GPU), a chip, a cloud based computing service or any suitable computing or computational device), an operating system 115, memory 120, executable code 125, storage 130, input devices 135 (e.g. a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet. Controller 105 may be configured to execute program code to perform operations described herein. The system described herein may include one or more computing device(s) 100.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Hash memory, a volatile memory, a non-volatile memory, a cache memory, a buffer, a short term memory unit, a long term memory unit, or other suitable memory units or storage units. Memory 120 may be or may include a plurality of, possibly different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein, for example, method of determining a condition of a subject based on VOCs in a gaseous phase, originating from the subject. In yet another example, code 125 may include a software application that performs a method of training a classification model 128 according to some embodiments of the invention. Although, for the sake of clarity, a single item of executable code 125 is shown in FIG. 1B, a system according to embodiments of the invention may include a plurality of executable code segments similar to executable code 125 that may be stored into memory 120 and cause controller 105 to carry out methods described herein.

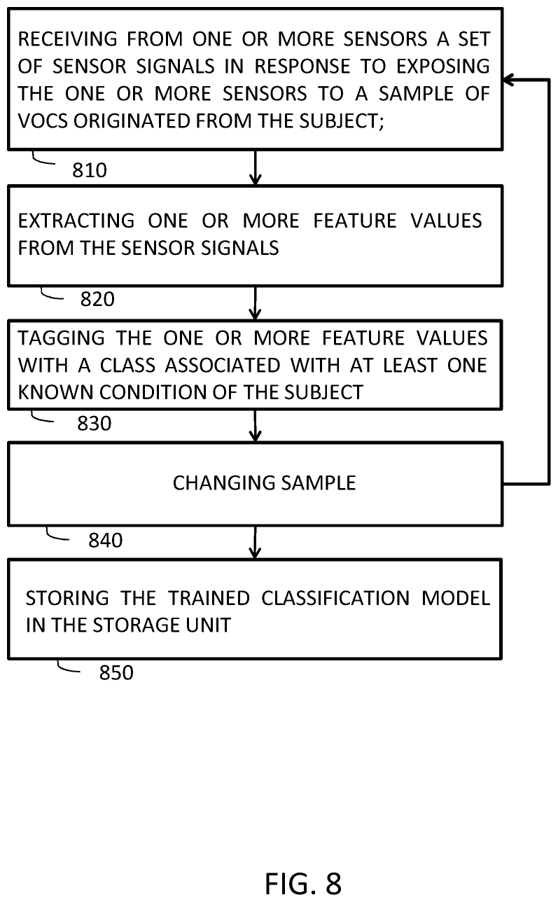

Memory 120 may further include a classification model 128 to be used in determining a condition of a subject according to some embodiment of the invention. A method of training classification model 128 is disclosed with respect to the flowchart of FIG. 8.

Storage 130 may be or may include, for example, a hard disk drive, a universal serial bus (USB) device or other suitable removable and/or fixed storage unit. In some embodiments, storage 10 may be a cloud based storing service communicating with system 10 over the internet. In some embodiments, some of the components shown in FIG. 1B may be omitted. For example, memory 120 may be a non-volatile memory having the storage capacity of storage 130. Accordingly, although shown as a separate component, storage 130 may be embedded or included in memory 120.

Input devices 135 may be or may include a keyboard, a touch screen or pad, one or more sensors or any other or additional suitable input device. Any suitable number of input devices 135 may be operatively connected to computing device 100. Output devices 140 may include one or more displays or monitors and/or any other suitable output devices. Any suitable number of output devices 140 may be operatively connected to computing device 100. Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105. Such a non-transitory computer readable medium may be for example a memory, a disk drive, or a USB flash memory, encoding, including or storing instructions, e.g., computer-executable instructions, which when executed by a processor or controller, carry out methods disclosed herein. The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to embodiments of the invention may include components such as, but not limited to, a plurality of central processing units (CPU), graphical processing units (GPUs), or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or maybe, for example, a personal computer, a desktop computer, a laptop computer, a workstation, a server computer, a network device, or any other suitable computing device.

In some embodiments, at least some of the components of computing device 100 may be assembled on a printed circuit board (PCB) to be assembled in a system such as system 10. In some embodiments, system 10 may further include a power supply 101 (illustrated in FIG. 1A) for providing electrical power to at least some of the components of system 10, for example, computing device 100, sensors 50, sensors 60 and the like. In some embodiments, power supply 101 may be assembled on the PCB.

A flowchart showing an example for operating the various component of system 10 is illustrated in FIG. 1C. To receive a signal from one or more sensors 50 a sample of VOCs may be introduced into system 10, thus a sampling cycle may be initiated in box 150. In some embodiments, controller 105 may control fan 33 to produce a flow of air that may carry the VOCs sample. The VOCs sample may be received directly from the air in the vicinity of system 10 or may be collected from an absorbing material (e.g., in a form of a ped) placed in a holder near the entrance to filler 31. Air flow produced by fan 33 may cause air that may include the sampled VOCs to pass through filter 31 to be filtered from particles and dust that may harm the measurements taken by one or more sensors 50. The air flow may than be introduced into manifold 23. In order to cause the air carrying the VOCs to be introduced into chamber 40, vacuum may be formed in chamber 40, by closing valve 34 (box 151) and opening valve 35 while operating pump 36 to pump the air from chamber 40 (box 153). By closing valve 35 and pump 36 and opening valve 34, the air carrying the VOCs sample may be introduced into chamber 40, following the closure of valve 34 (box 154).

In some embodiments, prior to the introduction the air carrying the VOCs sample into chamber 40, heating system such as the one included in regeneration system 70 may be operated (in box 160) in order to produce controlled environment and/or to clean sensors 50. In some embodiments, one or more sensors 50 may be exposed to the VOCs in the air for a predetermined amount of time and one or more sensor signals may be received by controller 105 (box 155). Following the sensing, valve 35 (box 156) and pump 36 (box 157) may be re-operated in order to pump up and clean chamber 40 (box 158). In some embodiments, the pumping of the air out of chamber 40 may also regenerate one or more sensors 50 to be cleaned from the previous VOCs prior to the introduction of an additional VOCs sample (box 159).

In some embodiments, different operation methods may be applied using system 10. For example, both valves 34 and 35 may stay open and the sensor signal may be generated by sensors 50 exposed to a flow of VOCs. In such operation method both fan 33 and pump 37 may constantly feed chamber 40 with a flow of air carrying VOCs.

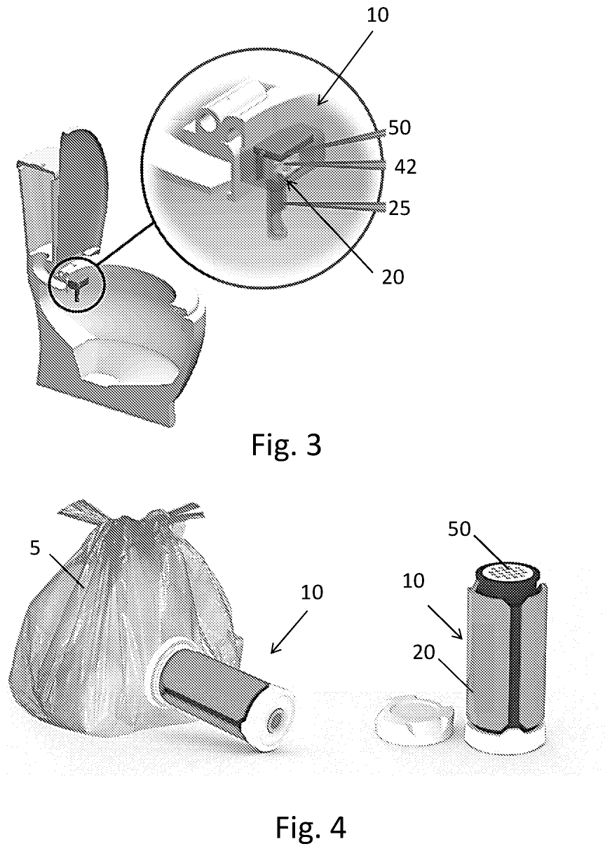

In some embodiments, system 10 may be housed in housing 20. Examples of different housing structures are illustrated and given in FIGS. 3 and 4. In some embodiments, housing 20 may include an intake (e.g., in a shape of a mouthpiece) configured to be inserted to a mouth of the subject or to receive a breath of air from the subject. The intake may be located before filter 31. In some embodiments, the breath of air may include VOCs from the subject's mouth and digestive system.

Reference is now made to FIG. 2A which is an illustration of an example of a chemi-sensor 50 according to some embodiments of the invention. Chemi-sensor 50 may be a resistor that may change its resistance upon exposure to chemicals in general and VOCs in gas phase in particular. Chemi-resistor may include a sensing element 52 made of metallic nanoparticles coated with organic ligands shell (illustrated in FIG. 2B) and metal electrodes (e.g., made Au or Pt) connecting elements 52 to the outside world. Sensing elements 52 and the metal electrode may be placed on a substrate 54, such as, semiconductor wafer with isolation layer made from glass, plastic etc.

Sensing element 52 may include a metallic nanoparticle core made from metals or alloys, for example, Au, Pt, Pd, Ag, Ni, Co, Cu, Al, Au/Ag, Au/Cu, Au/Ag/Cu, Au/Pt, Au/Pd, Au/Ag/Cu/Pd, Pt/Rh, Ni/Co, and/or Pt/Ni/Fe. The metallic nanoparticle core may be coated with organic ligand. Different organic ligand may be selected in order to sense different VOCs or families of VOCs. For example, organic ligands shells may consist of thiol (sulfide) bonding (to metal core) group such as: alkylthiols with C3-C24 chains, ω-functionalized alkanethiolates, arenethiolate, (γ-mercaptopropyl) tri-methyloxysilane, dialkyl disulfides, xanthates, oligonucleotides, polynucleotides, peptides, proteins, enzymes, polysaccharides, phospholipids and the like.

In some embodiments, system 10 may include an array (e.g., a plurality) of sensors 50. For example, such an array may include various sensors 50 each (or some) may be designed (e.g., by choosing the type of organic ligand) to be more sensitive to specific type of VOC or specific types of VOCs than to other VOCs (since some organic ligand can sense more than one type of VOC (e.g., a family of VOCs)). Accordingly, an array of sensors 50 may be configured to sense a plurality of VOCs.

Reference is now made to the graph illustrated in FIG. 2C which shows an example of a signal received from a sensor such as sensor 50 at different stages of the operation of system 10. Such a sensor signal may be produced by sensor 50 and sent to controller 105 for further analysis. At first when the system is in an ambient condition, only a small amount (relative for example, to the amount during sensing) of VOCs (e.g., there is always an amount of VOCs in the air) is present in chamber 50 (1), almost no signal (e.g., resistance in ohms) can be detected. Once an air carrying VOCs from the sample is introduced into camber 40 and exposed to sensor 50, the resistance may change (e.g., increase, as illustrated, decrease, alternate and the like) (2). At the end of the sensing/exposure time (3) the air carrying the VOCs is been pumped out of chamber 50 (4) and the signal is reduced to minimum (5) as the system returns to ambient conditions.

Reference is now made to FIG. 3 which is an illustration of a system 10 for determining a condition of a subject based on VOCs configured to be assembled in toilets. In some embodiments, the subject may be a human and the VOCs may be collected when the human uses the toilet. Such system 10 may be configured to sense the presence of the human in the toilet, for example, received from an external sensor, such as motion detector. When the human enters the restrooms the sensing sequence (e.g., boxes 151-159 of FIG. 1C) may be triggered. In some embodiments, the sensing sequence may be triggered after the human leaves the toilet or at a predetermined time after the human enters the toilet. VOCs 42 evaporated from urine and/or feces originated from the human may enter a column 25 to be pumped and flow towards a sensor array that includes two or more sensors 50. Housing 20 is configured to be attached to commercial toilets and to allow the collection of VOCs from the vicinity of system 10.

Reference is now made to FIG. 4 which is an illustration of a system 10 for determining a condition of a subject based on VOCs, configured to be assembled or included in a garbage bag 5. Garbage bag 5 may be configured to collect, for example, one or more dippers, hygienic pads and the like. Hosing 20 may be configured to hermetically be inserted into a hole in garbage bag 5. Upon closing of bag 5, a user may initiate or trigger the sensing sequence (e.g., boxes 151-159 of FIG. 1C). In some embodiments, in the configuration illustrated in FIG. 4 an array of sensors 50 is located at the portion of system 10 inserted into the interior of plastic bag 5 and the gas circulation system (not shown) is located in the portion of system 10 located outside bag 5.

Reference is now made to FIG. 5 which is an illustration of a pad 16 made from an absorbing material 15 for collecting VOCs from mammals, for example, livestock such as cows, horses, ships, etc. In some embodiments, absorbing material may be included in a pad 16 attached to the mammal by attachment means 17 (e.g., belts, stickers, etc.). In some embodiments, pad 15 may be configured to be attached to the mammal at various places, for example, the backside (under the tail as illustrated), on the back, near the briskets or the vagina (of a female mammal), on the forehead and the like. Absorbing material 15may be any material configured to absorb VOCs, for example, cotton, a polymeric sponge, a polymeric rubber, or any other suitable material. Absorbing material 15 may further be configured to release VOCs upon exposure to a flow of air, for example, at the entrance to system 10 (e.g., near filter 31). According to some embodiments, pad 16 may be placed in proximity to an intake of system 10 so that VOCs absorbed in absorbing material may be released and may enter chamber 40 upon operation of fan 33 and/or pump 37.

Reference is now made to FIG. 6 an illustration of an absorbing material 15 included in a pad for collecting VOCs from humans. In some embodiments, absorbing material 15 may be included in a bracelet 18, as illustrated, to be worn by the human. In some embodiments, absorbing material 15 may be integrated in a wearable device (e.g., a watch) or garment (e.g., a hat, a scarf, a sweatband and the like). According to some embodiments, absorbing material 15 may be attached to the human in any other way, such as for example, using a sticker. According to some embodiments, absorbing material 15 may be attached to a device or accessory carried regularly by the human, for example, a mobile phone a tablet computer and the like.

In some embodiments, system 10 may include a holder (not illustrated) for holding absorbing material 15 in the entrance to system 10 (e.g., before filter 31). Therefore, when gas circulation system 30 circulate air via filter 31, VOCs trapped by absorbing material 15 may evaporate and enter system 10 (e.g., via filter 31 and manifold 32) to be exposed to sensors 50, as disclosed with respect to FIGS. 1A-1C.

As should be understood by one skilled in the art the configurations illustrated in FIGS. 3-6 are given as examples only and the invention is not limited to these examples.

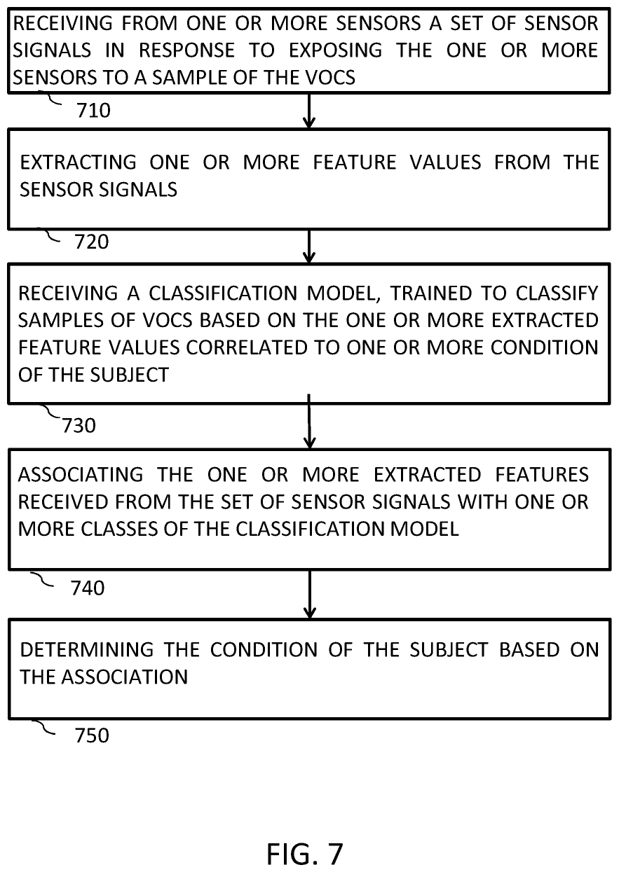

Reference is now made to FIG. 7 which is a flowchart of a method of determining a condition of a subject based on VOCs in a gaseous phase, originating from the subject according to some embodiments of the invention. The method of FIG. 7 may be performed by a controller such as controller 105 of system 10 of by any other suitable controller. In step 710, a set of sensor signals may be received, from one or more sensors, in response to exposing the one or more sensors to a sample of the VOCs. For example, a sample that includes VOCs originated from one or more subjects (e.g., a humans or mammals) may be introduced to circulation system 30 of system 10. For example, the sample may include air collected from the vicinity of a system 10 assembled in a toilet as illustrated in FIG. 3. In another example, the VOCs may be collected from a garbage bag 5 holding a dipper, as illustrated in FIG. 4. In yet another example, the VOCs may be collected from absorbing material 15, discussed herein above with respect to FIGS. 5 and 6.

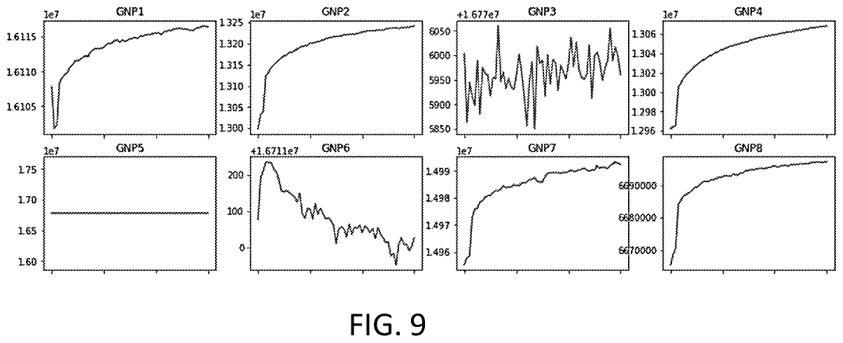

The VOCs included in the sample may be introduced into chamber 40 (e.g., VOCs 42) and to be sensed by one or more sensors 50, as discussed herein above with respect to FIG. 1C and FIGS. 2A-2C. In some embodiments, after the exposure a set of sensor signals may be received by controller 105. Such a set may include one or more sensor signals. An example for a set of 8 sensor signals from 8 sensors 50 is given in FIG. 9. In the example, given in FIG. 9, each signal was received from a sensor 50 sensitive to different types of VOCs.

In some embodiments, the sensor signal itself may not suffice for classifying the sample, since VOCs measurements cannot be scaled. Therefore, an analysis of one or more sensor signals in the set may be conducted. In step 720, one or more feature values may be extracted from the set of sensor signals. For example, each sensor signal may be analyzed and one or more feature values may be extracted. As used herein, feature values may be defined as any mathematical values that can be derived from analyzing a signal for example, the average value, the maximum value, the minimum value, the first time derivative, the second time derivative, Signal to noise ratio, incline gradient , decline gradient, rise time, overshooting value relative to steady state value, oscillation decay in time, oscillation frequency and the like. Accordingly, each signal may be associated with one or more feature values to be further evaluate by controller 105.

In step 730, a classification model (e.g., classification model 128) may be received, classification model 128 may be trained to classify samples of VOCs based on the one or more extracted feature values correlated to one or more condition of the subject. Classification model 128 may be stored and received from storage 130 or memory 120. A method for training and creating such a classification model is disclosed and discussed with respect to FIG. 8.

In step 740, the one or more extracted features received from the set of sensor signals may be associated with one or more classes of the classification model. In some embodiments, each class may be associated with a condition of the one or more subject, thus by identifying the class to which the one or more extracted features may be associated, the condition of the subject may be determine, in step 750.

In some embodiments, the condition is at least one of: a medical condition of the subject, a mental condition of the subject, an identity of the subject, a general wellbeing of the subject and the like.

For example, the subject may be a human using a toilet including system 10, as illustrated in FIG. 3. The VOCs collected when the human used the toilet may be detected by one or more sensors 50 and the set of signals may be analyzed by controller 105. For example, controller 105 may associate at least one feature or a group of feature values extracted with a class correlated with a medical condition of the human, for example, diabetes, cystitis, dehydration, lack of vitamins, flue, unbalanced diet, constipation, diarrhea, internal ingestion of foreign objects and the like. In some embodiments, the subject may be a female human and the conditions may include fertility that may be sensed in VOCs originated in the urine of the female human.

In another example, the one or more subject may include any female mammal and the condition may be fertility. The VOCs may be collected using an absorbing material, such as absorbing material 15 illustrated in FIGS. 5-6. In some embodiments, when a female mammal is ovulating the VOCs in the female mammal's urine change. These VOCs may be detected using the method disclosed and may be used to determine the hormonal cycle of the female mammal.

In yet another example, the one or more subjects may include at least two humans and the condition may be a chance for a successful matching. For example, VOCs samples taken from a couple (illustrated in FIG. 6) or from single humans may be used for finding if the couple or two individuals in a group have a high chance for a successful matching. A successful matching between couples may be defined as matching or pairing of people having in their relationship at least one positive indication that the relationship is a well-established one. In some embodiments, classification model 128 may include pairs of one or more feature values received from human pairs having at least one indication of having a successful matching. In some embodiments, the at least one indication may include at least one of: a relationship lasting more than a predetermined period, number of children, a reported affection and a reported sexual attraction.

In some embodiments, additional data may be included in classification model 128 and may be associated with classes in the model. For example, in the case of making a successful matching additional data such as: gender, age, sexual preferences, ethnicity, culture, lifestyle, diet and the like may be considered when classifying pairs having a high chance for successful matching. This type of data may include a subject related data and may add to the accuracy of the determination of the chance of having a successful matching. In some embodiments, additional subject related data such medical condition, mental condition and general wellbeing may further be included and associated with one or more classes of the classification model. In some embodiments, the subject related data may be received from a user (e.g., the subject, a caregiver and the like) using a user interface, for example, input device 135. In some embodiments, the subject related data may be provided to system 10 by a caregiver (e.g., a parent) using for example, an application running on a mobile device communicating with computing device 100 (e.g., via the internet) that may upload data into controller 105.

In some embodiments, the subject related data may include data related to a specific sample taken form the subject. For example, if the subject is a baby and the sample was taken from a dipper of the baby, as illustrated and discussed with respect to FIG. 4, the subject related data may include the time period at which the baby wore the dipper, the sleeping time while wearing the dipper and the like. Such data may be provided to system 10 by a caregiver (e.g., a parent) using for example, an application running on a mobile device that may upload data into controller 105.

In some embodiments, the additional data may include sample related data. Such sample related data may include the environmental condition at which the sample was taken, for example, received from one or more sensors 70. For example, sample related data may include, the humidity level in chamber 40, temperature in chamber 40 or an ambient temperature, geographic location at which the sample was taken, time and date at which the sample was taken and the like.

In some embodiments, parameters such as the geographic location and the time and date may affect the type and amount of VOCs originated form subject. For example, in warmer locations, were people sweat more often than in colder places, the VOCs may include sweat originated VOCs. Similarly, the date (e.g., season or the exact weather at this date) and time may also affect the VOCs.

In some embodiments, the method may further include receiving one or more initial signals from one or more sensor 50, when the sensors are not exposed to a VOC sample. The initial signals may be associated as surrounding background signals that may be used to filter background noise from the set of sensor signals using the initial signals.

In some embodiments, the method of FIG. 7 may be used to identify the subject. Therefore, the condition may be the identity of the subject. In some embodiments, classification model 128 may be trained to identify features which are typical for a specific subject regardless of any changes in the subject's in location, diet, medical condition, etc. Such a training method is discussed herein below with respect to the method of FIG. 8.

In some embodiments, controller 105 may further be configured to display to the subject or a user associated with the subject commercial data related to the determined condition. For example, controller 105 may be configured to display on a mobile device of the parent uploading data related to the dipper, babies related commercials. In another example, controller 105, after successfully matching of two individuals, may display to the two individuals commercial data related to romantic hotels or restaurants.

Reference is now made to FIG. 8 which is a flowchart of a method of training a classification model 128 according to some embodiments of the invention. The method of FIG. 8 may be performed by a controller such as controller 105 of system 10 of by any other suitable controller. Steps 810 and 820 may be substantially the same as steps 710 and 720 of the method of FIG. 7. In step 830, the one or more feature values may be tagged with a class associated with at least one known condition of the subject. In some embodiments, the known condition may be received from a user or users via a user interface, for example, input device 135 or an application running on a mobile device associated with the user and communicating with computing device 100. For example, a subject known to have an abnormal medical condition, for example, diabetes, may provide a sample to be sensed an inspected by system 10. Accordingly, one or more feature values extracted from the provided sample may be tagged with the class “having diabetes”.

In some embodiments, additional data related to the subject may also be received and tagged with the one or more feature values, for example, age, gender, lifestyle, weight, height, ethnicity, diet, geographical location and the like. For example, the class, to which the provided sample from diabetic subject was tagged, may further include the following tagging: male, normal BMI, Asian, a US residence, age 60-70 years, smoking and not conducting physical activity. In step 840, steps 810-830 may be repeated with a new sample.

In some embodiments, the subject related data may be received from an additional sensor, for example, a sensor that may directly measure the chemical composition of the urine sample. Such as sensor may provide, for example, the level of fertility hormones in the urine, and may provide a direct indication to the fertility of the female providing the urine sample. Additional sensors may include a thermometer to measure the subject's body temperature, oxygen saturation sensor, blood pressure sensor, heartrate sensor and the like. In some embodiments, the related data may include medical records of the subject provided form the subject healthcare provider. The medical records may be received by controller 105, via the internet, form an external database associated with the healthcare provider.

In some embodiments, an additional data related to the sample, or the condition at which the sample were sensed may also be provided, for example, form a sensor such as sensor 70, as disclosed herein above with respect to the method of FIG. 7. All the collected data may be associated and tagged into a class in the classification model. For example, a class may include human females at ages 30-40 with overweight and fertility problems.

In some embodiments, controller 105 may be configured to receive extracted feature values from samples taken from a “control group”. In some embodiments, classification model 128 may further include samples received from an additional group of subjects having similar characteristics but not having the known condition, for example, human females at ages 30-40 with overweight not having any fertility problems. In some embodiments, controller 105 may compare feature values extracted form the additional group of subjects with the classified group of subjects, in order to eliminate feature values which are similar in both groups due to the similar characteristics, thus leaving in the class only feature values originated from the known medical condition.

An example for training classification model 128 to identify a subject may include extracting a plurality of features from the set of signals each associated with a sample taken from a specific subject under different conditions, for example, at different geographical locations, at various times and dates, after a change in a diet, after a change in the medical condition and the like. In some embodiments, the classification model may be trained to look for feature values that may be found in the majority of samples taken form the subject, regardless of the different conditions at which the samples were taken. These feature values may be tagged with the identity of the subject and may further use to identify the subject.

In another example, classification model 128 may be trained to determine a chance of having a successful matching between two humans. In the training process samples from couples that were identify as having at least one indication for successful matching may be collected. For example, samples from couples having relationships lasting form more than 15 years, couples having at least 2 children, couples reporting mutual affection and couples reporting mutual sexual attraction, may be collected. The couples may further be tagged with additional tagging such as, heterosexual couples, lesbian couples, gay couples, according to age groups, ethnicity, residence location and the like. In some embodiments, classification model 128 may be trained to look for similar features extracted from signals received from samples provided by the couples, that may indicate “a successful matching”. These features may be identified in all couples having “a successful matching” or in sub-groups. For example, classification model 128 may identify similar features in signals received from lesbian couples, having a relationship that last at least 10 years, having 2 children, 20-40 years old and living in Italy.

In yet another example, classification model 128 may be trained to determine the general wellbeing of babies based on samples received from dippers (as discussed hereinabove with respect to FIG. 4). In some embodiments, classification model 128 may receive form a caregiver, via an application running on a mobile device, information related to the general wellbeing of the baby providing the dipper. For example, each sample originated from a dipper may be tagged with: how many hours the baby sleep uninterruptedly at night, is he/she teething, does the baby suffers from gases, age, gender, diet and the like. In some embodiments, classification model 128 may be trained to identify similar features in signals received from samples given form babies ages 4-7 month, teething and sleeping uninterruptedly less than 3 hours.

In step 840, classification model 128 may be stored in memory 120 and/or storage unit 130.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of determining a condition of a subject based on Volatile Organic Compounds (VOCs) in a gaseous phase, originating from the subject, the method comprising:
receiving from one or more sensors a set of sensor signals in response to exposing the one or more sensors to a sample of the VOCs;
extracting one or more feature values from the set of sensor signals;
receiving a classification model, trained to classify samples of VOCs based on the one or more extracted feature values correlated to one or more condition of the subject;
associating the one or more extracted features received from the set of sensor signals with one or more classes of the classification model; and
determining the condition of the subject based on the association.

2. The method of claim 1, wherein the condition is at least one of: a medical condition of the subject, a mental condition of the subject, an identity of the subject and a general wellbeing of the subject.

3. The method of claim 1 or claim 2, further comprising:
receiving additional data ;
associating the received additional data with one or more classes of the classification model; and
determining the condition of the subject also based on the additional data association.

4. The method of claim 3, wherein the additional data comprises sample related data.

5. The method of claim 4, wherein the sample related data comprises at least one of: humidity level, temperature, geographic location at which the sample was taken, time and date.

6. The method of claim 3, wherein the additional data comprises subject data.

7. The method of claim 6, wherein the subject related data comprises at least one of: gender, age, medical condition, ethnicity, culture, lifestyle and diet.

8. The method of claim 6, wherein the subject related data comprises data related to a specific sample taken form the subject.

9. A method according to any one of claims 1-8, wherein the VOCs are collected by at least one of:
an absorbing material attached to the at least one subject, an absorbing material attached to a device carried by the at least one subject and a container for collecting VOCs evaporating from the at least one subject.

10. A method according to any one of claims 1-9, wherein the at least one subject is a mammal.

11. The method of claim 10, wherein the VOCs comprises VOCs included in a at least one of: urine of the subject, sweat of the subject and saliva of the subject.

12. A method according to any one of claims 1-7, wherein the subject is a human and the method further comprises:
collecting a VOCs sample when the human uses a toilet; and
extracting the VOCs from the sample and exposing the one or more sensors to the extracted VOCs.

13. A method according to any one of claims 1-9, wherein the at least one subject is a female mammal and the condition is fertility.

14. The method of claim 13, wherein the VOCs comprises VOCs included in at least one of: urine, sweat and saliva of the female mammal

15. The method of claim 13, wherein the VOCs comprises VOCs included in the at least one of: skin and hair of the female mammal.

16. A method according to any one of claims 1-9, wherein the at least one subject includes at least two humans and wherein the condition is a chance for a successful matching.

17. The method of claim 16, wherein the classification model comprises pairs of one or more feature values received from humans' pairs having at least one indication of having a successful matching.

18. The method of claim 17, wherein the at least one indication comprises at least one of: a relationship lasting more than a predetermined period, number of children, a reported affection and a reported sexual attraction.

19. A method according to any one of claims 1-9, wherein the at least one subject is a human baby and the condition is general wellbeing.

20. The method of claim 19, wherein the VOCs comprises VOCs included in at least one of: urine and feces.

21. A method according to any one of claims 1-9, wherein the at least one subject is a mammal and the condition is an identity of the mammal

22. A method according to any one of the preceding claims, further comprising:
receiving one or more initial signals from the one or more sensor;
associating the initial signals as surrounding background signals; and
filtering background noise from the set of sensor signals using the initial signals.

23. A system for determining a condition of a subject based on Volatile Organic Compounds (VOCs) in a gaseous phase, originating from the subject, the system comprising:
one or more VOCs sensors configured to detect VOCs originated from the at least one subject; and
a controller configured to:
receive from the one or more sensors a set of sensor signals in response to exposing the one or more sensors to a sample of the VOCs;
extract one or more feature values from the sensor signals;
receive a classification model, trained to classify samples of VOCs based on the one or more extracted feature values correlated to one or more condition of the subject;
associate the one or more extracted features received from the set of sensor signals with one or more classes of the classification model; and
determine the condition of the subject based on the association.

24. The system of claim 23, further comprises:
a chamber for holding the one or more sensors; and
a gas circulation system for directing VOCs in a gas phase towards the one or more sensors.

25. The system of claim 24, wherein the gas circulation system includes at least one of: a fan, a pump, one or more gas monitoring sensors, and one or more valves.

26. A system according to any one of claims 23-25, further comprising a regeneration device for regenerating the one or more sensors.

27. The system of claim 26, wherein the regeneration device comprises at least one of: a heating element, a vacuum pump and a stream of gas.

28. A system according to any one of claims 23-27, further comprising one or more additional sensors for detecting a condition of the at least one subject.

29. A system according to any one of claims 23-28, further comprising a holder for holding an absorbing material carrying the VOCs collected from the at least one subject.

30. The system of claim 29, wherein the absorbing material comprises an absorbing material configured to absorbed VOCs from the subject.

31. A system according to any one of claims 23-30, wherein the one or more sensors include one or more chemi-resistors comprising metallic nanoparticles coated with organic ligands shell, metal oxide sensor (MOS), catalytic near IR sensor, photoionization detector (PID), IR open path sensor, portable gas-chromatography mass spectrometer (GC-MS) and electro-chemical sensor.

32. A system according to any one of claims 23-31, wherein the controller is configured to carry out any one of the methods of claims 1-19.

33. A method of training a classification model to determine a condition of a subject, the method comprising:
a. receiving from one or more sensors a set of sensor signals in response to exposing the one or more sensors to a sample of VOCs originated from the subject;
b. extracting one or more feature values from the sensor signals;
c. tagging the one or more feature values with a class associated with at least one known condition of the subject; and
d. repeating steps (a) through (c) with a different sample, to train the classification model.

34. The method of claim 33, wherein the condition is at least one of: a medical condition of the subject, a mental condition of the subject, an identity of the subject and a general wellbeing of the subject.

35. The method of claim 33 or claim 34, further comprising:
receiving an additional data; and
tagging the additional data with the class associated with the known condition of the subject.

36. The method of claim 35, wherein the additional data comprises sample related data.

37. The method of claim 36, wherein the sample collecting data comprises at least one of: humidity level, temperature, geographic location at which the sample was taken, time and date.

38. The method of claim 35, wherein the additional data comprises subject related data.

39. The method of claim 38, wherein the subject related data comprises at least one of: gender, age, medical condition, ethnicity and diet.

40. The method of claim 38, wherein the subject related data comprises data related to a specific sample taken form the subject.

41. A method according to any one of claims 33-40, wherein the VOCs are collected by at least one of:
an absorbing material attached to the at least one subject, an absorbing material attached to a device carried by the at least one subject and a container for collecting VOCs evaporating from the at least one subject.

42. A method according to any one of claims 33-41, further comprising:
receive one or more initial signals from the one or more sensor;
associate the initial signals as surrounding background signals; and
filter background noise from the set of sensor signals using the initial signals.

43. A system for training a classification model to determine a condition of a subject, the system comprising:
one or more VOCs sensors configured to detect VOCs originated from the at least one subject;
a storage unit; and
a controller configured to:
a. receive from one or more sensors a set of sensor signals in response to exposing the one or more sensors to a sample of VOCs originated from the subject;
b. extract one or more feature values from the sensor signals;
c. tag the one or more feature values with a class associated with at least one known condition of the subject;
d. repeat steps (a) through (e) with a different sample, to train the classification model; and
e. store the trained classification model in the storage unit.

44. The system of claim 43, wherein the controller is configured to:
receive at least one known condition of the subject for each sample.

45. The system of claim 44, further comprising a user interface and wherein the controller is configured to:
receive at least one known condition from the user interface.

46. The system of claim 44, further comprising at least one additional sensor and wherein the controller is configured to:
receive at least one known condition from a signal received from the additional sensor.

47. The system of claim 46, wherein the additional signal is indicative of the at least one condition.

48. The system according to any one of claims 43-47, wherein the controller is configured to carry out any one of the methods of claims 33-42.
